(19) United States
(12) Patent Application Publication
(10) Pub. No.: US 2025/0239365 A1
(43) Pub. Date: Jul. 24, 2025

(54) SYSTEM AND METHOD FOR INTELLIGENT PATIENT ASSESSMENT

(71) Applicant: Ralph G. Kuechle, Ph.D., Orange, CA (US)

(72) Inventor: Ralph G. Kuechle, Ph.D., Orange, CA (US)

(21) Appl. No.: 18/825,824

(22) Filed: Sep. 5, 2024

Related U.S. Application Data
(63) Continuation-in-part of application No. 18/379,897, filed on Oct. 13, 2023, now abandoned.

(51) Int. Cl. G16H 50/20; A61B 5/16; G16H 10/20; G16H 80/00

(52) U.S. Cl. CPC G16H 50/20; A61B 5/165; G16H 10/20; G16H 80/00

(57) ABSTRACT

An intelligent automated patient assessment system comprising a system processor; software stored on a tangible medium operationally coupled to the system processor; and a user interface operationally coupled to the processor whereby, in response to the user interface, the processor executes the software to automatically output patient assessment results in response to patient inputs. In the illustrative embodiment, the processor is programmed with a natural language processor coded for automated sentiment detection. The system detects and processes neutral, positive and negative 10 sentiment for automated mood detection. Mood detection is then incorporated into an assessment provided to a clinician. The system further includes code for activating an alarm in response to detected negative sentiment.

### BACKGROUND OF THE INVENTION

### Field of the Invention

The present invention relates to computers and computing systems and methods. More specifically, the present invention relates to systems and methods for automated mental health assessment and assistance with diagnosis.

### Description of the Related Art

Mental health concerns in the U.S. are growing. According to the Substance Abuse and Mental Health Services Administration (SAMHSA), 17.9% or 43.4 million US adults aged 18 or older experience mental illness. In addition to the substantial percentage of U.S. adults that struggle with mental health, the importance of receiving treatments is increasing. According to data from the National Institute of Mental Health (NIMH), anxiety disorders are the most common type of mental illness, with 18.1% of US adults suffering from a variety of anxiety disorders. These mental disorders include panic disorder, obsessive-compulsive disorder, posttraumatic stress disorder, and all types of phobias.

Mood disorders are also quite common and can frequently co-occur with anxiety disorders. Mood disorders include major depressive disorder, dysthymic disorder, cyclothymic disorder, and bipolar disorder; the NIMH estimates that 9.5% of Americans over the age of 18 suffer from these types of mental illnesses. Other mental illnesses that industry professionals provide counseling for include eating disorders, disruptive behavior disorders, varied levels of autism, and personality disorders. Since many of these mental disorders occur in conjunction with substance abuse problems, industry professionals, especially mental health professionals and substance abuse social workers, are often counseling patients on several different issues simultaneously.

According to Statista, 22.1% of individuals ages 18 to 25, transitional age youth (TAY), have reported suffering from mental illness while 21.2% of adults 26 to 49 report struggling with mental illness. In addition, more females than males struggled with mental illness. In addition, Statista reports that millennials are more likely to utilize mental health services if available through employers. In fact, 44% of millennials would utilize an emotional health app, 36% would utilize a telephonic consultation, and 39% would utilize on online visit.

Hence, more and more companies and individuals are putting resources towards decreasing the negative stigma associated with receiving mental health services, creating increased traction for easily accessible mental health solutions.

The psychology industry includes mental health practitioners that diagnose and treat mental, emotional and behavioral disorders. The nature of the industry enables it to thrive even when economic conditions are poor. Mental health services are needed especially when there is higher potential for damage to individuals' ego, strength and emotional stability.

The psychology industry has grown strongly over the past five years. Mental health services are needed regardless of economic conditions, which insulate the industry from economic fluctuations. For instance, although the number of people with private health insurance fell and the unemployment rate spiked during the recession, lowering per capita disposable income, the industry continued to grow. Increased demand for industry services resulting from the high stress of living in tough economic times, in combination with increased federal funding for Medicare and Medicaid services, offset the negative effects of the declining number of Americans with private insurance.

Industry performance relies on several conflicting trends. For instance, job loss and financial stresses can lead to depression and anxiety for individuals dealing with these struggles, boosting demand for psychologists and other mental health professionals. However, these circumstances often coincide with declines in the number of people with private health insurance, which indicates a decrease in demand for industry services. Moreover, the economic crisis prompted many state governments to cut mental health funding.

Despite these cuts, demand for industry services has remained high. The latest data from the Substance Abuse and Mental Health Services Administration (SAMHSA) indicate that the percentage of the population with serious psychological distress increased over the past five years. Additionally, practitioners reported that there was an increase in the number of people seeking treatment.

Clinical psychologists are concerned with the assessment, diagnosis, treatment and prevention of mental disorders. While some clinical psychologists specialize in treating severe psychological disorders, such as schizophrenia and depression, many others may help people deal with personal issues, such as a divorce or the death of a loved one. Often, clinical psychologists provide clients with an opportunity to discuss personal issues and offer an alternative perspective to interpreting and understanding those issues. Psychologists train in a variety of approaches to assist clients, often determined by their chosen field of specialty. Areas of specialization within psychology include clinical psychology, health psychology, neuropsychology, and child psychology.

Demand for psychologists industry services is influenced by the psychological condition of the population and the awareness of the importance of mental health. The psychological condition is, in turn, influenced by many factors, including but not restricted to: the state of the economy; perception of sensory input; mood as manifested by depression and anxiety levels; cultural factors; birth and genetic defects; accidents involving head injuries (including from road accidents); substance abuse; and age (dementia). These factors can be measured by the Consumer Confidence index, the number of physician visits and marriage statistics, among other indicators.

Other factors influencing demand for practitioners' services include: the availability and use of public and private health insurance for behavioral health conditions and the extent to which insurance covers specific conditions; the level of real household income (people with higher incomes tend to spend more on healthcare and are more likely to have private health insurance); the cost and availability of industry services; and the availability and cost of alternative services.

Mental health prevention and care programs can have positive effects on mental health and can reduce demand for mental health services. In addition, the development of new forms of psychotherapy and psychosocial treatments has created new options for offering effective care and support for people with mental illness. There is evidence that for certain types of illnesses, pharmaceutical treatments can act as substitutes (at least partially) for psychotherapy. Prozac, for example, can reduce the effects of depression.

Mental health issues can occur at any time, but studies suggest that recent tough economic times sparked a new wave of mental illness in the United States. People can feel hopeless and out of control during economic downturns due to job loss, wage reductions or home foreclosure. A recent national survey by the National Alliance on Mental Health and Depression is Real Coalition found that unemployed people are four times more likely to experience depression and other mental health issues than people who are employed and are also four times more likely to consider suicide than those with full-time jobs. People who are employed but have experienced pay cuts or reduced hours are twice as likely to suffer from depression or severe mental illness and are five times more likely to report feeling hopeless most or all of the time, than people who have not experienced this type of change at work. Anxiety, depression, other mental illness, or general feelings of hopelessness are large reasons why individuals seek mental health industry services. Therefore, tough economic times that spark emotional and mental stress tend to increase demand for mental health services.

There is an increased understanding of the benefits in providing early intervention for children with emotional disturbances. The extent to which society understands mental illness and drug dependence influences demand for care required for individuals with these conditions. Awareness is on the rise, due in part to industry associations promoting mental health causes and a national mental health awareness month. Awareness is important to affecting industry demand, because public and private spending on mental health services is influenced by the appreciation of the economic benefits of treating mental illness and substance abuse problems (e.g., increase in productivity, reduction in workdays lost, lower spending on physical illness and on welfare benefits). Furthermore, rising awareness decreases the stigma surrounding seeking help for mental ailments, further boosting demand for industry services.

Hence, there is a need in the art for an intelligent patient assessment and diagnostic platform that may be utilized by clinical psychologists and other mental health professionals to diagnose a greater number of individuals who might benefit from a mental health treatment program but may not otherwise utilize mental health services due to cost, stigma, and/or a lack of time.

### SUMMARY OF THE INVENTION

The need in the art is addressed by the intelligent patient assessment system of the present invention. The invention seeks to provide easier and less expensive access to clinical psychologists and other mental health professionals by creating a telehealth application that better analyzes patients' mental health symptoms.

In the illustrative embodiment, the patient assessment system comprises a system processor; software stored on a tangible medium operationally coupled to the system processor; and a user interface operationally coupled to the processor whereby the processor is programmed to output an automated patient assessment in response to patient inputs. In the best mode, the processor is programmed to serve as a chatbot with a natural language processor coded for automated sentiment detection. The system detects and processes neutral, positive and negative sentiments for automated mood detection. Mood detection is then incorporated into an assessment provided to a clinician. The system further includes code for activating an alarm in response to detected negative sentiment.

### BRIEF DESCRIPTION OF THE DRAWINGS

FIG. 1 is a high level block diagram of an illustrative embodiment of a LooperRoom system and method for intelligent patient assessment in accordance with the present teachings.

FIG. 2 is a block diagram showing a patient or clinician user's system operationally coupled to a system for intelligent patient assessment in accordance with a networked embodiment of the present invention.

FIG. 3 is a flow diagram that illustrates the method and operation of intelligent patient assessment in accordance with the illustrative embodiment of the present invention.

FIG. 4 is a flow diagram that illustrates a patient's interaction with a LooperRoom system and method for intelligent patient assessment in accordance with the illustrative embodiment of the present invention.

FIG. 5 is a flow diagram that illustrates a clinician's interaction with the LooperRoom system and method for intelligent patient assessment in accordance with the illustrative embodiment of the present invention.

FIG. 6 is a flow diagram that illustrates a clinician's interaction with patients using the illustrative embodiment of the LooperRoom system and method for intelligent patient assessment in accordance with the present teachings.

FIGS. 7a-9b are screenshots illustrative of data output by the dashboard of the LooperRoom system and method for intelligent patient assessment in accordance with the present teachings.

FIG. 10 is an illustration implementation of a decision tree by Medium.

FIG. 11 shows a portion of a decision tree created in accordance with the teachings of the present invention.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

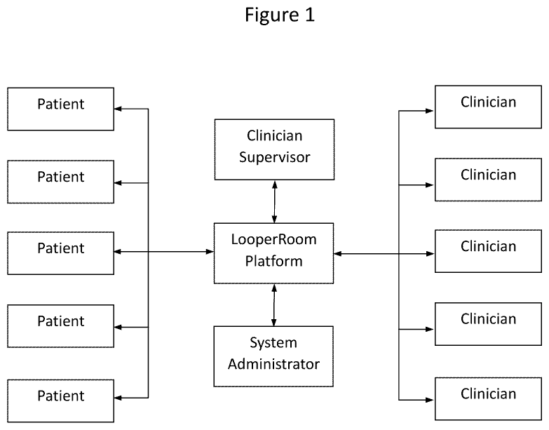

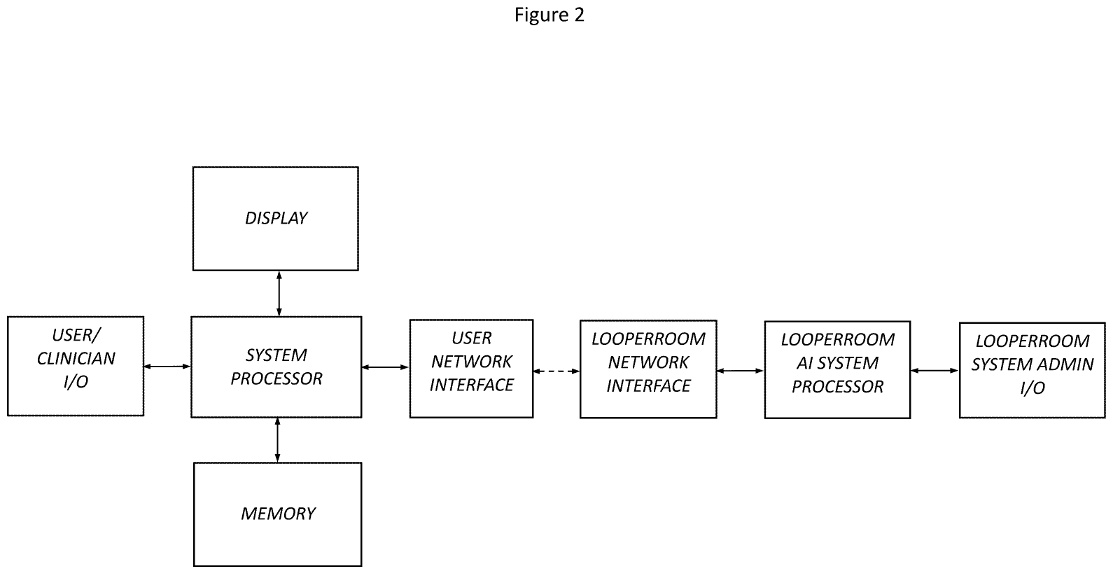

FIG. 1 is a high level block diagram of an illustrative embodiment of a system and method for intelligent patient assessment in accordance with the present teachings. FIG. 1 shows that multiple patients and multiple clinicians connect to a LooperRoom platform through their own individual platforms as discussed more fully below (see FIG. 2).

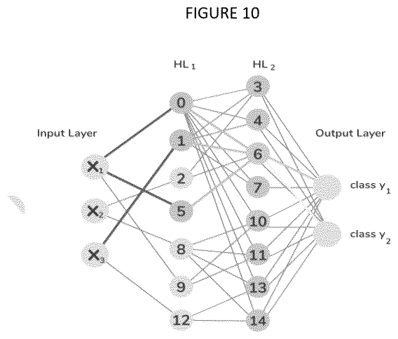

In accordance with the present teachings, as a first step, a novel automated conversational agent or ‘chatbot’, hereinafter referred to as a ‘LooperRoom Chatbot’ or ‘the Chatbot’, is set up and programmed as follows:
1. Formulate a set of queries from the Chatbot to a patient:
   At this an initial step, in the illustrative application, a group of experienced psychologists is assembled to provide a set of queries from the bot to a typical patient to initiate a session including an initial greeting such as “Hello (patient's name) how are you today?” In the best mode, this step should be thorough. In the illustrative implementation, this step consumed more than 500 man-hours.
2. Create a Chatbot decision tree based on anticipated patient responses as follows:
   A. Make a list of all statements that could be anticipated from a patient in response to each prompt at the initial stage of a typical psychotherapy session.
   B. Make a list of typical clinician responses to each statement made by the patient in response to the initial prompt.
   C. Continue the simulated interaction until a complete decision tree is created as illustrated in FIG. 10 below.
3. Next, program a set of nodes, or decision points, to provide a software based embodiment of the decision tree, using conventional software programming techniques, based on the anticipated patient responses and best Chatbot responses provided by the skilled clinicians at Step 2B above. In the illustrative embodiment, Juji was used to create the decision tree. (See https://juji.io/juji-platform/“https://juji.io/juji-platform/).
4. Test the functionality of the decision tree by psychologists by using the chatbot. The decision tree is completed when the chatbot gives appropriate responses without errors as a clinician would.
5. Run the program, collect patient responses and update each patient's profile with each patient's response to each subsequent simulated prompt from a typical clinician.
6. Perform a sentiment detection here and after every patient response during which responses to the Chatbot are analyzed for polarity, i.e., positive, negative or neutral.
7. Following each chatbot response, allow the conversation to continue until the patient decides to stop talking about a topic, changes the topic, or ends the chat. In the illustrative embodiment, the system includes code to enable the patient to end the Chatbot session at any time by pressing a “That's All” button, by discontinuing to respond to the Chatbot and/or logging out of the LooperRoom app.
8. The chatbot responds appropriately to the patient's input based on its programming as set forth above and elaborates as necessary until the process is complete.
9. The decision tree is then updated with the actual prompts and responses.

In accordance with the illustrative implementation, a Large Language Model (LLM) was used to develop the LooperRoom decision tree. However, the present invention is not limited to the use of a word based LLM. For example, a logic-based model such as Aigo's Integrated Neuro-Symbolic Architecture (INSA) could also be utilized in the LooperRoom decision tree without departing from the scope of the present teachings. See https://aigo.ai/

Thus, in accordance with the present teachings, the system is set up with an interface to allow access by clinicians, a clinician supervisor, and a system administrator to each of their platforms. Those of ordinary skill in the art will appreciate that the system is not limited to a client server architecture. That is, one or more patients and/or one or more clinicians may access the LooperRoom platform directly through a mobile application or with a simple keyboard, mouse and display if such functionality is enabled by the system administrator.

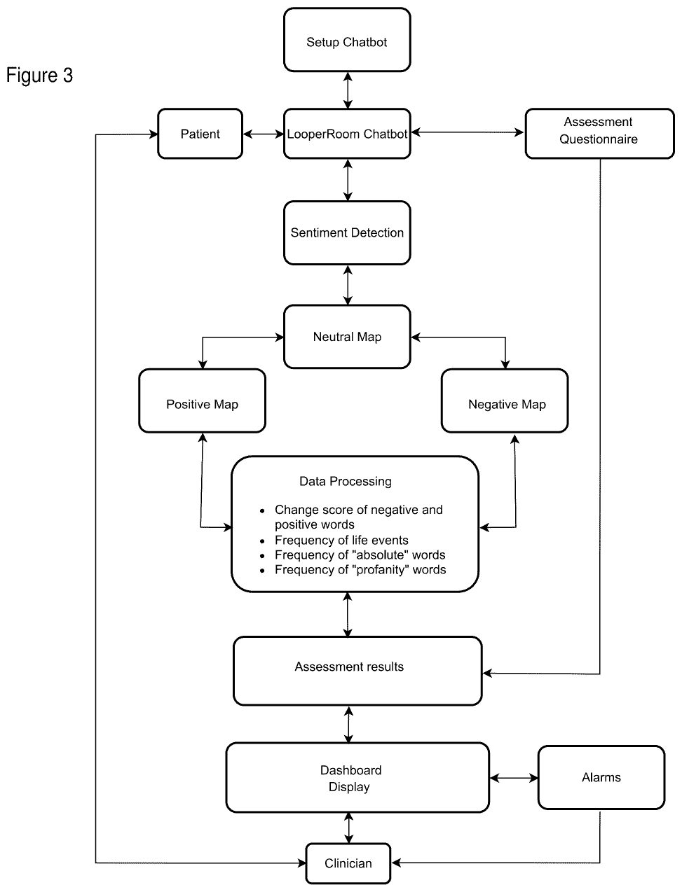

FIG. 2 is a simplified block diagram showing a patient or clinician user's system operationally coupled to the system for intelligent patient assessment of FIG. 1 in accordance with a networked embodiment of the present invention. As shown in FIG. 2, each clinician or patient's platform includes a system processor, display, memory and user input/output (I/O) interface. The I/O interface typically includes a mobile application, or a keyboard, a mouse, and a display, however, the display is shown separately in FIG. 2. The user/clinician's system processor is coupled to the LooperRoom platform (shown as a LooperRoom artificially intelligent (AI) system processor) via a user's network interface, a local area network, wide area network and/or the internet and a LooperRoom network interface. The user's platform and network interface as well as the LooperRoom network interface may be of conventional design and construction. The operation of the LooperRoom AI system processor is illustrated in the flow diagram of FIG. 3.

FIG. 3 is a flow diagram that illustrates the method and operation of intelligent patient assessment in accordance with the illustrative embodiment of the present invention.

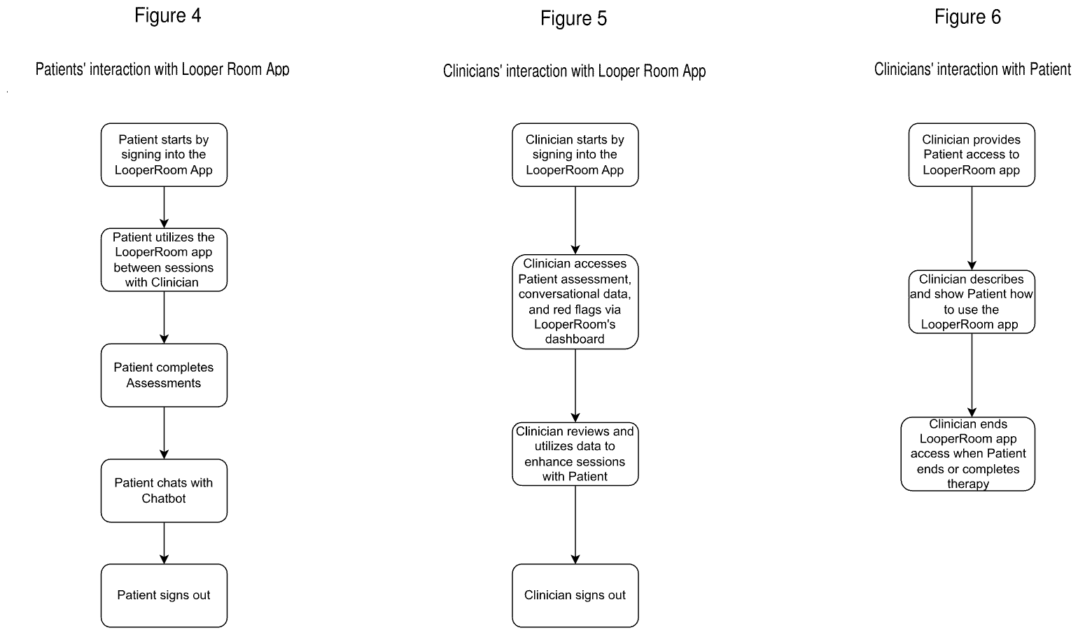

FIG. 4 is a flow diagram that illustrates a patients' interaction with a LooperRoom system and method for intelligent patient assessment in accordance with the illustrative embodiment of the present invention.

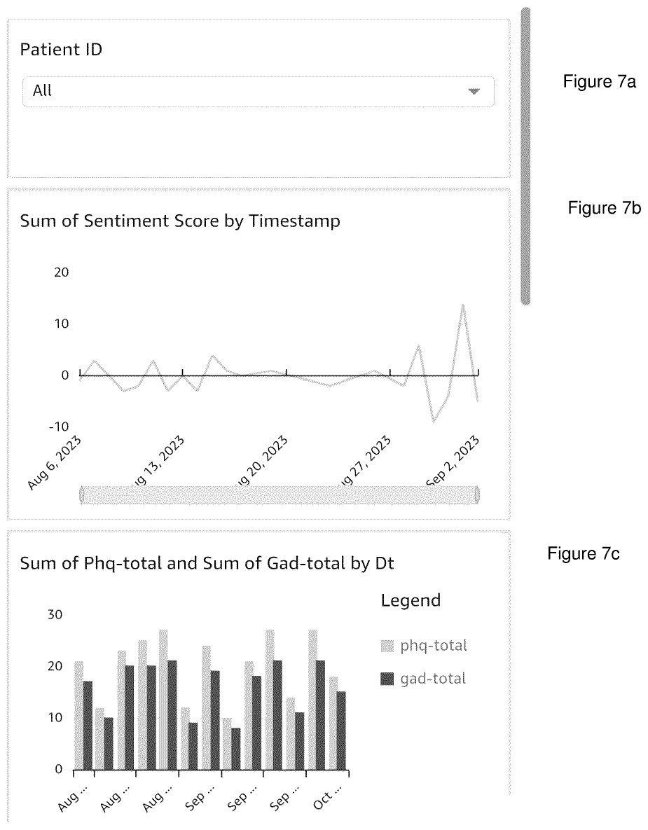

FIG. 5 is a flow diagram that illustrates a clinicians' interaction with the LooperRoom system and method for intelligent patient assessment in accordance with the illustrative embodiment of the present invention.

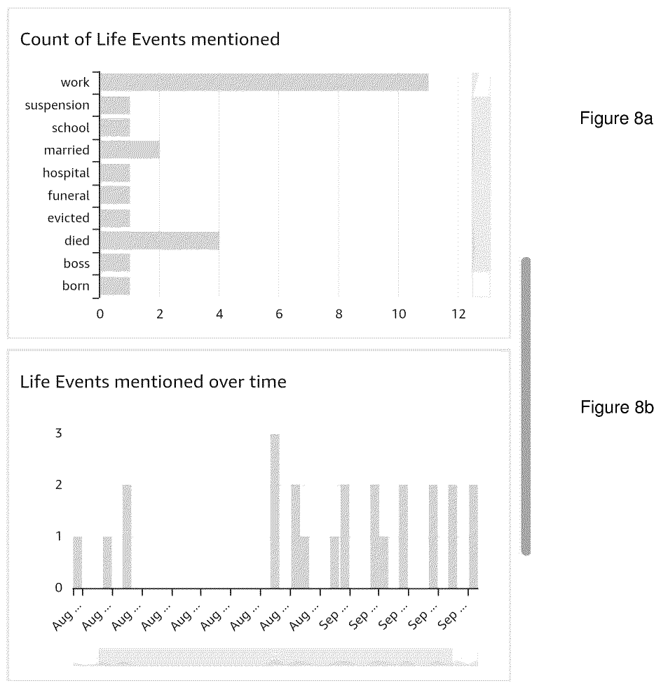

FIG. 6 is a flow diagram that illustrates a clinicians' interaction with patients using the illustrative embodiment of the LooperRoom system and method for intelligent patient assessment in accordance with the present teachings.

As shown in FIG. 3, the LooperRoom app connects with an off-the-shelf chatbot such as the Juji platform to set up a chat interface for the LooperRoom Chatbot (i.e., a conversation flow, where conversation flow means the chatbot interacts with a patient). Those of ordinary skill in the art will appreciate that the present invention is not limited to use with the Juji tool. Numerous AI chatbots may be used to set up, train and/or initialize the LooperRoom chatbot to function in the manner discussed more fully below without departing from the scope of the present invention.

Next, clinicians provide patients with access to the LooperRoom app (that includes gamification components). In this context, ‘gamification components’ include a reward system such as awards (digital badges) as recognition for consistently using the Chatbot on a daily basis. A clinician sends each patient an access code that the patient uses to create a user account. The access code automatically links the patient to the clinician's dashboard within the LooperRoom platform.

Next, each patient completes a weekly anxiety (GAD-7) and depression (PHQ-9) assessment before obtaining access to LooperRoom Chatbot. Both the GAD-7 and the PHQ-9 are well-known in the art. GAD-7 and PHQ-9 are valid and reliable assessment instruments for anxiety (GAD-7) and depression (PHQ-9), backed by extensive scientific research. GAD-7 and PHQ-9 are widely used by Clinicians across the U.S.A.

In accordance with the present teachings, patients chat with the LooperRoom Chatbot to verbalize their thoughts and feelings, share their experiences and challenges, and get feedback from the Chatbot. In the best mode, software and hardware are included in the system to enable the patient to interact with the LooperRoom Chatbot using various methods of communication including: texting, speech, video and immersive presence with video and speech with Virtual Reality (VR) in real time with associated audio.

In accordance with a preferred embodiment of the invention, with the addition or utilization of a web camera, microphone and speakers between the patient and the Chatbot along with code for voice and facial recognition including code for mood, gesture and/or posture detection.

The patient's mood may be determined by assessing tonality of the voice (i.e., raising voice, staying calm, being assertive) and facial expressions during interactions and etc. Sentiment analysis is utilized for emotion detection (how the patient feels).

Speech with video in VR may be implemented using silhouette that the patient utilizes to interact with the Chatbot such as that provided by IVN (www.ivn.net) as disclosed and claimed in U.S. Pat. No. 11,218,669 issued Jan. 4, 2022 and entitled System and Method For Extracting And Transplanting Live Video Avatar Images, the teachings of which are hereby incorporated herein by reference.

As an alternative, the patient can choose a conventional avatar for the Chatbot.

As shown in FIGS. 3-6, LooperRoom tools are utilized and customized to detect various target sentiments (positive, negative, neutral) to guide the conversation of the Chatbot and the patients to various routes, such as the LooperRoom Neutral, Positive, and/or Negative Map throughout the conversation. For sentiment, LooperRoom utilizes a sentiment detection algorithm, such as Juji's built in tool, with added customizations that specify/recategorize certain works as: positive, negative, neutral, somewhat positive, or somewhat negative etc.

In the best mode, emotion analysis is utilized to support sentiment analysis utilizing custom code, FaceReader (see https://www.noldus.com/applications/emotion-analysis) or Real-time Facial Emotion Detection using deep learning (as published on GitHub https://github.com/atulapra/Emotion-detection). This embodiment uses more advanced machine learning techniques to analyze more complex emotions like fear, anger, sadness, love, frustration, and many more. Both sentiment analysis and emotion analysis facilitate the process of analyzing patients' feelings.

Next, conversational data are processed. In the illustrative embodiment, this is achieved by using a serverless, event-driven compute service that cleans, manages, and organizes data such as AWS Lambda.

By way of example, the assessment results include the score change between the frequency of negative and positive words used; frequency of life events mentioned (e.g., divorce, retirement, moving, etc.); frequency of “absolute” words used (e.g., always, never, every, etc.); frequency of profanity words used, etc.

Next, the assessment results are sent to a tool to create data visualizations, along with results from anxiety (GAD-7) and depression (PHQ-9) assessments. This tool may be implemented with AWS Quicksight or other suitable software. In the illustrative embodiment, the data visualization tool imports the data from the output of the AWS Lambda function. These data are stored in data visualization tool. Then the data visualization tool creates a visualization through analysis of the data.

The analysis is a template for how the data are displayed and visualized on the dashboard. The dashboard uses the template of the analysis and applies the data to the graphs of the dashboard and connects them to the assigned patients. The dashboard is designed to visualize the data in a way that maintains certain security requirements allowing a Clinician only to see his/her assigned patients

In the illustrative embodiment, the data visualizations are shown as graphs and are sent to the dashboard for viewing by a clinician. In the best mode, the LooperRoom dashboard also includes a “keyword search feature” where clinicians can search for any keyword and the app will pull up conversational texts that include that word.

If patients use “red flag” words (e.g., I am going to hurt myself) this will send an alert to the clinician via the app. The Chatbot will remind patients that the Chatbot is not designed to directly help in this situation and will provide links for support and urging patients to reach out to emergency contacts.

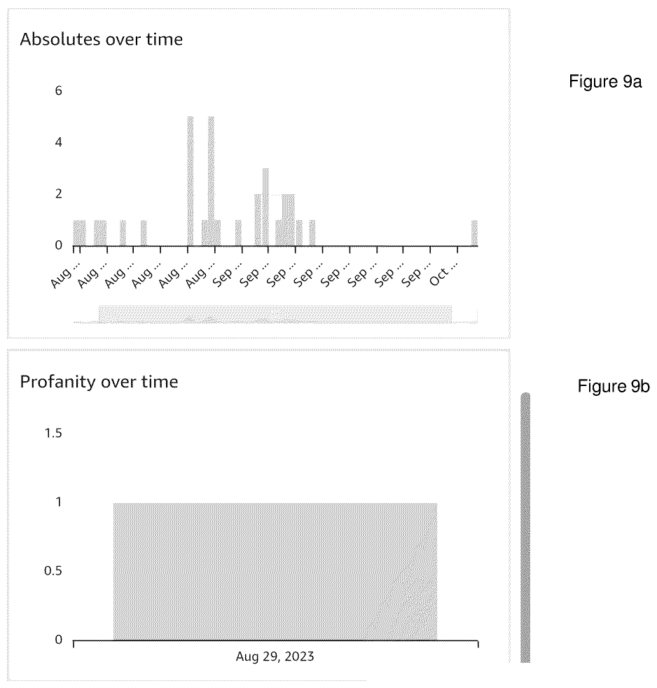

FIGS. 7a-9b are screenshots illustrative of data output by the dashboard of the LooperRoom system and method for intelligent patient assessment in accordance with the present teachings. As illustrated in the screenshots of FIGS. 7a-9b, clinicians have access to the data through the LooperRoom Dashboard to enhance their sessions with their patients.

FIG. 7a shows the upper section of the clinician dashboard where the clinician can choose from a drop-down menu the patient he/she wants to see on the dashboard. Clinician can also choose “All” to look at all patients combined to monitor the overall progress of all patients combined.

FIG. 7b shows the graph for sentiment detection meaning it visualizes how the patient feels over time. Clinician can choose time frames for sentiment detection such as current week, current month, last several months, or sentiment detection since start of treatment.

FIG. 7c shows the graph for scores of the PHQ-9 and GAD-7 assessments over time. Clinician can choose time frames for scores of the PHQ-9 and GAD-7 assessments such as current week, current month, last several months, or since start of treatment.

FIG. 8a shows the graph for the count of life events mentioned by patient during conversations with Chatbot. As such, it is a summary of all life events.

FIG. 8b shows the graph for life events mentioned over time during conversations with Chatbot allowing a Clinician to choose time frames for life events mentioned over time during conversations with Chatbot such as current week, current month, last several months, or since start of treatment.

FIG. 9a shows the graph for scores of absolutes over time. Clinician can choose time frames for scores of absolutes over time such as current week, current month, last several months, or since start of treatment.

FIG. 9b shows the graph for scores of profanity over time. Clinician can choose time frames for scores of profanity over time such as current week, current month, last several months, or since start of treatment.

FIG. 10 is an illustration implementation of a decision tree as published at by Medium.com at https://medium.com/@navarai/decision-trees-vs-neural-networks-ff46f47ce0a0.

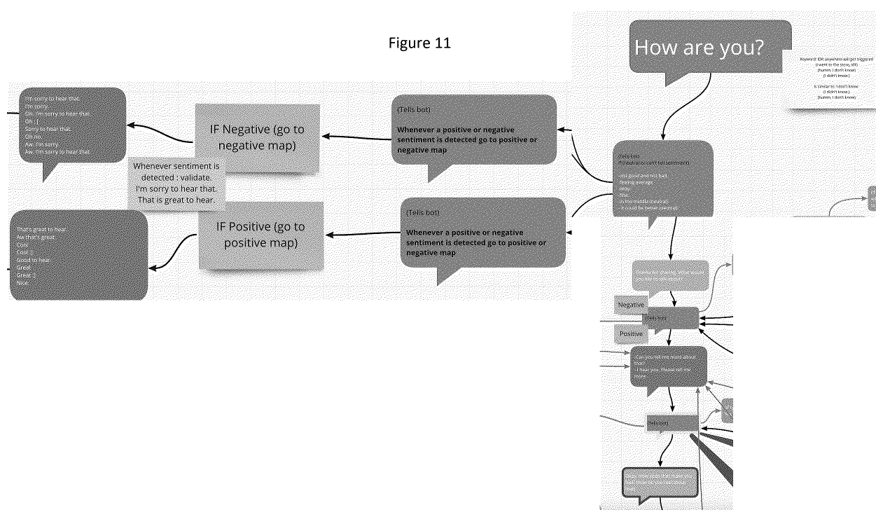

FIG. 11 is an illustration of a portion of a LooperRoom decision tree implemented in accordance with the present invention. The blue boxes in FIG. 11 contain the initial greeting and is the location at which the chatbot determines whether the patient response is positive, neutral, or negative. The orange boxes represent the outcome of that determination and lead the Chatbot to the responses on the positive, neutral, or negative path. The green boxes contain the responses utilized by the Chatbot. There are several options for any response to enable the Chatbot's communication flow to be more natural and less redundant.

Data collection, analysis, and visualization on the LooperRoom dashboard is a key feature of the present invention. The LooperRoom dashboard is an information management tool that receives data from a linked database to provide data visualizations. The LooperRoom dashboard enables Clinicians to monitor clinical progress of their patients over time in real time via a system with a dashboard that is instantly and continually updating changes. In the illustrative embodiment, two tools are used to implement the dashboard: 1) a data analysis tool such as AWS Lambda or any other suitable data analysis program and 2) a visualization platform such as AWS Quicksight or any other suitable program for visualization of data on the dashboard. The inventive Chatbot collects the data. The data analysis tool does the data analyses and the visualization platform displays the data results on the dashboard. Thus, the inventive Chatbot provides high-level information in one view that end users can use to answer a single query.

Thus, the present invention provides a system and method for collecting data regarding a patient, providing an assessment, and providing the data and assessment to a clinician for diagnosis and treatment. The AI based system of the present invention enables clinicians to measure treatment progress over time, collaborate with their patients on treatment progress and potential adjustment to treatment interventions, as well as treatment goals and objectives, and thereby improve the relationship between clinician and patient: aka the ‘therapeutic alliance’. The stronger the therapeutic alliance is, the more likely the treatment is or will be effective as there is a positive correlation between those two variables. Hence, the system and method of the present invention may be expected to strengthen the therapeutic alliance and, as a result, improve the effectiveness of treatment. In addition, it should make the clinician's workflow more efficient. Both efficiency of workflow and effectiveness of treatment should positively impact the clinician's anxiety and stress level and therefore, reduce the clinician's risk for burnout.

Those having ordinary skill in the art and access to the present teachings will recognize additional modifications, applications and embodiments within the scope thereof. For example, in a mobile application, the inventive platform provides a variety of features connecting users with resources including mental health professionals such as the Centralized Assessment Team (CAT) and the National Suicide Lifeline (988).

It is therefore intended by the appended claims to cover any and all such applications, modifications and embodiments within the scope of the present invention.

1. An intelligent patient assessment system comprising:
a system processor;
software stored on a tangible medium operationally coupled to the system processor and programmed to provide a chatbot with a decision tree operative to interact with a patient and provide an assessment of a patient, said software including code for translating commands collected from a group of professional practitioners as to how a qualified practitioner would conduct a session with a patient into said decision tree; and
a user interface operationally coupled to the processor including a camera and a microphone
whereby, in response to inputs from the patient, the processor executes the software to automatically detect and analyze complex emotions including fear, anger, sadness, love, or frustration and output patient assessment results in response thereto.

2. The invention of claim 1 wherein said user interface includes a dashboard.

3. The invention of claim 2 wherein said dashboard includes a data analysis tool.

4. The invention of claim 3 wherein said dashboard includes a data visualization program.

5. The invention of claim 1 wherein the software includes code for enabling the processor to serve as a natural language processor.

6. The invention of claim 5 further including code for sentiment detection.

7. The invention of claim 6 further including code for neutral, positive and negative mood detection.

8. The invention of claim 7 wherein said assessment is provided in response to said mood detection.

9. The invention of claim 8 wherein said assessment is provided to a clinician in response to said sentiment detection.

10. The invention of claim 5 further including code for activating an alarm in response to detected sentiment.

11. The invention of claim 1 wherein the chatbot is initialized with a conversation flow matrix.

12. The invention of claim 1 wherein the chatbot administers a questionnaire to a patient.

13. The invention of claim 12 further including code for sentiment detection.

14. The invention of claim 12 further including code for neutral, positive and negative mood detection.

15. The invention of claim 14 wherein said assessment is provided in response to sentiment detection and said mood detection.

16. The invention of claim 15 wherein said assessment is provided to a clinician in response to said sentiment detection and mood detection.

17. The invention of claim 12 further including code for activating an alarm in response to detected negative sentiment.

18. An intelligent patient assessment system comprising:
a system processor;
software stored on a tangible medium operationally coupled to the system processor and programmed by professional clinicians to provide a chatbot with a decision tree operative to interact with a patient in a professional manner and provide a professional assessment of a patient; and
a user interface operationally coupled to the processor whereby, in response to the user interface, the processor executes the software to automatically output patient assessment results in response to patient inputs.

19. An intelligent patient assessment method including the steps of:
providing a system processor;
providing software stored on a tangible medium operationally coupled to the system processor and programmed by professional clinicians to provide a chatbot with a decision tree operative to interact with a patient in a professional manner and provide a professional assessment of a patient;
providing a patient interface operationally coupled to the processor whereby, in response to the user interface, the processor executes the software to automatically output patient assessment results in response to patient inputs via text, audio or video; and
activating the processor to execute the software to provide an automated patient sentiment assessment in response to patient inputs via said interface.
